(19) United States
(12) Patent Application Publication
(10) Pub. No.: US 2005/0019781 A1
(43) Pub. Date: Jan. 27, 2005

(54) Methods for diagnosing dementia-related neurological disorders

(76) Inventors: Carsten Buhmann, Hamburg (DE); John Jacob Hemperly, Apex, NC (US); Ralf Kleene, Hamburg (DE); Melitta Schachner, Hamburg (DE); Helen Strekalova, Hamburg (DE); Tomas Muller-Thomsen, Barum (DE)

Correspondence Address: CASTELLANO MALM FERRANO & BUCK PLLC, 2121 K STREET, NW, SUITE 800, WASHINGTON, DC, 20037, US

(21) Appl. No.: 10/645,930

(22) Filed: Aug. 22, 2003

Related U.S. Application Data
(60) Provisional application No. 60/405,206, filed on Aug. 22, 2002.

(51) Int. Cl. C12Q 1/68; G01N 33/53; G01N 33/567

(52) U.S. Cl. 435/6; 435/7.2

(57) ABSTRACT

The current invention relates to methods for diagnosing and monitoring the progression of a dementia-related neurodegenerative disease in a patient suspected of having such a disease by determining the levels of at least one cell adhesion molecule.

### BACKGROUND OF THE INVENTION

1. Field of the Invention

The current invention relates to methods for diagnosing and monitoring the progression of dementia-related neurological disorders in a patient suspected of having such a disease by determining the levels of at least one cell adhesion molecule.

2. Background of the Invention

The increasing number of patients with Alzheimer's Disease and other dementias, in part due to the overall aging of the population, is an important health problem for the affected individuals, their caregivers, and the public health system. These diseases are currently diagnosed and treated by experienced clinicians, but there are few ante-mortem biochemical measures of their onset and severity. Clearly a more objective tool for accessing these diseases would be invaluable.

Cell adhesion and recognition molecules are very important to the proper development and functioning of the nervous system. They are found in all vertebrate species and are highly conserved. Two of these molecules, L1 and NCAM, are cell surface glycoproteins found on many types of neurons and glial cells. Both molecules comprise multiple isoforms that differ, for example, in their modes of attachment to the cell surface, the structure of their intracellular domains, and the presence or absence of particular amino acid sequences that arise by alternative splicing of messenger RNAs. In addition, both molecules vary in the amount of covalently bound carbohydrates that in turn can influence their binding activities and interaction with other molecules on the cell surface. For example, some NCAM molecules contain high levels of an unusual carbohydrate, polysialic acid, and are designated as PSA NCAM. In addition to mediating cell binding, both L1 and NCAM are capable of conveying signals from external influences into intracellular signaling pathways.

Because of their biological importance, the amounts and types of L1 and NCAM may be particularly involved in human neurological diseases. For example, mutations in L1 have been linked to a common form of developmental defects and mental retardation (referred to as the MASA syndrome, a genetic disease linked to mutations in L1 and marked by mental retardation, adducted thumbs, suffling gait and aphasia), and may be the most prevalent cause of the “idiopathic” mental retardation among institutionalized individuals. Levels of L1, NCAM, and an alternatively spliced VASE (Variably Alternatively Sliced Exon) isoform of NCAM have been found to differ in individuals with schizophrenia. The levels may differ both in cerebrospinal fluid (CSF) and postmortem tissues. Because of these observations, we have developed sensitive, specific and quantitative assays for L1 and NCAM in the CSF and assayed their concentrations in a variety of neurological diseases. The levels of both molecules appear to be elevated in Alzheimer's Disease and in certain types of dementia, and decreased in patients with multiple sclerosis.

### SUMMARY OF THE INVENTION

The current invention provides methods of diagnosing and/or monitoring the progression of dementia-related neurological disorders in a patient exhibiting symptoms of and being suspected of having such a disease. The methods comprise obtaining a sample from a patient and determining the levels of at least one cell adhesion molecule selected from the group consisting of L1 and neural cell adhesion molecule (NCAM) and making a diagnosis based on these determined levels, as compared to normal patients.

### BRIEF DESCRIPTION OF THE DRAWINGS

FIG. 1A is a graph demonstrating the range of measured L1 concentrations in individuals diagnosed with various forms of dementia, using the methods described herein. Specifically, the values (circles) represent L1 concentration in the cerebrospinal fluid (CSF) of patients with Alzheimer disease (AD), vascular dementia (VD), dementia of mixed type (MT), multiple system atrophy (MSA), Parkinson disease (PD), diffuse Lewy body dementia (DLBD), epilepsy, amyotrophic lateral sclerosis (ALS), polyneuropathy (PNP), multiple sclerosis (MS) and normal controls (NC). Each horizontal line represents the mean value of L1 concentration. FIG. 1B depicts the mean values of L1 concentration±standard error.

FIG. 2A is a graph demonstrating the range of measured NCAM concentrations in individuals diagnosed with various forms of dementia, using the methods described herein. Specifically, the values (circles) represent NCAM concentration in the cerebrospinal fluid (CSF) of patients with Alzheimer disease (AD), vascular dementia (VD), dementia of mixed type (MT), multiple system atrophy (MSA), Parkinson disease (PD), diffuse Lewy body dementia (DLBD), epilepsy, amyotrophic lateral sclerosis (ALS), polyneuropathy (PNP), multiple sclerosis (MS) and normal controls (NC). Each horizontal line represents the mean value of NCAM concentration. FIG. 2B depicts the mean values of the concentration of NCAM±standard error.

FIG. 3A is a graph demonstrating the range of measured PSA-NCAM concentrations in individuals diagnosed with various forms of dementia, using the methods described herein. Specifically, the values (circles) represent PSA-NCAM concentration in the cerebrospinal fluid (CSF) of patients with Alzheimer disease (AD), vascular dementia (VD), dementia of mixed type (MT), multiple system atrophy (MSA), Parkinson disease (PD), diffuse Lewy body dementia (DLBD), epilepsy, amyotrophic lateral sclerosis (ALS), polyneuropathy (PNP), multiple sclerosis (MS) and normal controls (NC). Each horizontal line represents the mean value of PSA-NCAM concentration. FIG. 3B depicts the mean values of the concentration of PSA-NCAM ± standard error.

FIG. 4A depicts the mean values of the concentration of L1±standard error in the dementia and non-dementia groups. FIG. 4B depicts the mean values of the concentration of NCAM±standard error in the dementia and non-dementia groups. FIG. 4C depicts the mean values of the concentration of PSA-NCAM±standard error in dementia and non-dementia groups.

FIG. 5A depicts the mean values of the concentration of L1±standard error in patients with neurodegenerative and non-degenerative disorders. FIG. 5B depicts the mean values of the concentration of NCAM±standard error in patients with neurodegenerative and non-degenerative disorders. FIG. 5C depicts the mean values of the concentration of PSA-NCAM±standard error in patients with or without neurodegeneration.

### DETAILED DESCRIPTION OF THE INVENTION

The current invention provides methods of diagnosing dementia-related neurological disorders in a patient exhibiting symptoms of and suspected of having such a disease. The methods comprise obtaining a sample from a patient and determining the levels of at least one cell adhesion molecule selected from the group consisting of L1 and neural cell adhesion molecule (NCAM), and making a diagnosis based on these determined levels, when compared to normal patients.

As used herein, a dementia-related neurological disorder is disease characterized by the presence of dementia. As used herein “dementia” is determined using standard clinical procedures, with the degree of dementia being defined by the score in the Mini Mental State Examine (MMSE), as detailed in Folstein M. F., Folstein S. E. and McHugh P. R., J Psychiatry Res., 12:189-198 (1975). As used herein, dementia includes all degrees of clinical dementia, from mild to moderate to severe dementia. For example, a score of 30 to 27 points in the MMSE is classified as non-demented, a score of 26 to 20 is considered mildly demented, a score of 19 to 10 points is considered moderately demented and a score of 9 to 0 points is considered severely demented. Dementia, as used herein, includes all ranges of scores of the MMSE, except, of course, those scores classified as non-demented. However, “dementia,” as used herein, is not to be limited by the presence or absence of an MMSE score.

The dementia-related neurological disorder that is diagnosed or monitored is any disease or condition that affects or is affected by the central or peripheral nervous systems. The neurological disorder can be degenerative or non-degenerative. A degenerative neurological disorder is used to mean degeneration of any of the cells of the central or peripheral nervous system. Cell types of the central and peripheral nervous systems include, but are not limited to, neurons and glial cells. Types of neurons include, but are not limited to, dorsal root ganglion cells, bipolar cells, pyramidal cells, spinal motor cells and Purkinje cells. Types of glial cells include, but are not limited to, oligodendrocytes, Schwann cells and astrocytes. Furthermore, degeneration of cells of the nervous system includes the complete cessation (cell death) or partial cessation of cellular metabolic activity. This cessation could be apoptotic in nature, i.e., programmed cell death, or the degeneration could be necrotic in nature, such as from a toxic or biological insult to the cells. Examples of neurodegenerative diseases include, but are not limited to, Alzheimer's Disease, dementia of mixed type, Parkinson's Disease, diffuse Lewy Body dementia, Multiple System Atrophy, amyotrophic lateral sclerosis (ALS), progressive supranuclear palsy, and Pick disease. Examples of nondegenerative neurological disorders include, but are not limited to, vascular dementia, multiple sclerosis, epilepsy, polyneuropathy, normal pressure hydrocephalus, psychosis, alcoholism, major depression, paraneoplastic encephalopathy, rheumatoid arthrosis, epidural hematoma, varicella zoster neuritis and spinal muscle atrophy.

As used herein, the terms “subject” or “patient” are used interchangeably and are intended to mean an animal, preferably a mammal, including a human. Furthermore, “patient” is also intended to include a subject that exhibits symptoms of the dementia-related neurological disorder. The symptoms can be expressed, genetically, phenotypically and/or behaviorally. For example, the subject suspected of having a dementia-related neurological disorder may possess a genetic mutation or disorder that is genetically associated with a dementia-related neurological disorder. Similarly, the patient may have abnormal levels of a particular protein or other compound that is indicative or often used in the diagnosis of a neurological disorder. Likewise, the patient may exhibit defined behavioral symptoms indicative or often used in the diagnosis of a neurological disorder.

The methods of the current invention involve obtaining a sample from a patient. The sample should comprise cerebrospinal fluid (CSF). Thus, the sample may include additional components or additives that are naturally occurring or are synthetic, or the sample may be pure CSF. The CSF may be processed after it is obtained. Such examples of processing include, but are not limited to, concentrating, diluting, purifying, or admixing the obtained CSF.

As used herein, “obtain” can be any means whereby one comes into possession of the sample. Thus, obtain is used mean collection and/or removal of the sample from the patient. Examples of obtaining a sample from a patient are readily apparent and include, but are not limited to lumbar puncture procedures (spinal tap). Furthermore, “obtain” is also used to mean where one receives the sample from another who was in possession of the sample previously.

The current invention comprises determining the levels of at least one cell adhesion molecule. As used herein, a cell adhesion molecule is a molecule that affects the adhesiveness of cells either to other cells, i.e., cell-cell adhesion molecules (CAMs), or to substrates or surfaces, i.e., cell-substrate adhesion molecule (SAMs). The major classes of cell adhesion molecules include the integrins, selectins, cadherins and members of the immunoglobulin (Ig) superfamily. Each of the classes of cell adhesion molecules include several, well-known members that are widely recognized in the art. Additionally, new cell adhesion molecules may be identified by one of several ways, including but not limited to, interactions with antibodies known to disrupt cell adhesion, immunoprecipitation, cloning of putative CAMs or SAMs from expression libraries, binding or competition assays, conjugation of the putative cell adhesion molecule to microspheres, attachment assays, centrifugal-force assays and transfection experiments. Examples of known cell adhesion molecules include, but are not limited to, E-cadherin, P-cadherin, N-cadherin, B-cadherin R-cadherin, EP-cadherin, OB-cadherin, M-cadherin, cadherin-5, cadherin-12, protocadherin 43, desmocollin 1, desmoglein 1, α₁β₁integrin, α₂β₁integrin, α₃β₁integrin, α₄β₁integrin, α₅β₁integrin, α_{V}β₁integrin, α_{L}β₂integrin, α_{M}β₂integrin, α_{V}β₃integrin, P-selectin, E-selectin, intercellular adhesion molecule 1 (ICAM 1), ICAM 2, neural cell adhesion molecule (NCAM), platelet-endothelial cell adhesion molecule (PECAM), vascular adhesion molecule (VCAM), carcinoembryonic adhesion molecule and L1.

In one embodiment of the current invention, the levels of L1 are determined for the purposes of diagnosing the dementia-related neurological disorders. In another embodiment, the levels of neural cell adhesion molecule (NCAM) are determined for the purposes of diagnosing the dementia-related neurological disorders. The invention encompasses assaying for all isoforms of NCAM, including, but not limited to, NCAM 105-115, NCAM-120, NCAM-140 and NCAM-180. In yet another embodiment, the levels of both L1 and NCAM are determined for the purposes of diagnosing the dementia-related neurological disorders.

The levels of at least one cell adhesion molecule are determined in the methods of the current invention. As used herein, “determining the levels” is used to mean performing a quantitative assay to reveal a quantity of the particular cell adhesion molecule being assayed. The quantity may be a raw number, an absolute or relative quantity, a ratio between the measured amounts of cell adhesion molecule(s) and another measured value such as an internal standard or another cell adhesion molecule or other component of the assayed sample. Additionally, the raw data may be used in a more complex algorithm to arrive at the “determined levels.” Furthermore, the determined levels could be an average (arithmetic mean, media, mode) of data points or the determined levels could be derived from n=1.

The methods of the current invention require a comparison of the determined levels with another set of data. This additional set is generally derived from at least one “normal” individual that is not exhibiting symptoms of, or suspected of having any aspect of a dementia-related neurological disorder as described herein. The additional set of data may be derived from the same individual now suspected of having a dementia-related neurological disorder, or the additional data can be derived from a different normal individual. The additional data may represent the average (arithmetic mean, media, mode) of levels determined from one or a population of two or more normal individuals.

To diagnose a subject suspected of having a dementia-related neurological disorder using the methods described herein, the determined levels of the cell adhesion molecule must be greater than the levels found in a normal individual.

The current invention also provides methods of monitoring the progression of dementia-related neurological disorders in a patient exhibiting symptoms of and suspected of having such a disease. The methods comprise obtaining at least two samples from a patient suspected of having a dementia-related neurological disorder and determining levels of at least one cell adhesion molecule selected from the group consisting of L1 and neural cell adhesion molecule (NCAM) in each of the at least two samples. The determined levels are compared to one another, and any difference(s) in the at least two determined levels will indicate a progression in the dementia-related neurological disorder.

As used herein, “progression” is used to mean a worsening of the symptoms of the dementia-related neurological disorder, or it can mean an alleviation of the symptoms of the dementia-related neurological disorder. In other words, “progression” is temporal in nature and is thus used to in conjunction with improvements in the patient's symptoms, as well as a deterioration or maintenance of the patient's symptoms.

Thus the methods of monitoring of the progression of the dementia-related neurological disorder described herein can be used to monitor the efficacy of a particular therapeutic regimen designed to combat the symptoms or cause of the dementia-related neurological disorder, or it may be used to monitor the progression in a patient not undergoing therapy.

Monitoring the progression of the dementia-related neurological disorder comprises comparing the determined levels of the cells adhesion molecules in the at least two obtained samples. No difference(s) in the determined levels of the cell adhesion molecule indicates that the patient's condition or symptoms may not be worsening since the previous determination. However, an increase in the determined levels of the cell adhesion molecule over time would indicate that the patient's condition or symptoms might be worsening with time. Alternatively, a decrease in the determined levels of the cell adhesion molecule over time would indicate that the patient's condition or symptoms might be improving with time.

Comparing the determined levels may be accomplished in a variety of ways including, but not limited to, arithmetic subtraction, producing a ratio of the determined levels or using the determined levels in more complex algorithms. The method of comparison need only highlight any existing differences in the determined levels.

In at least one embodiment, the current invention involves the use of a monoclonal antibody, NCAM 14.2, directed against human NCAM, which can be used in any of the methods of the current invention. The NCAM 14.2 antibody can recognize all known isoforms of NCAM, including NCAM 105-115, NCAM-120, NCAM-140, NCAM-180 as well as the polypeptides of α2,8-polysialic acid-containing NCAM (PSA-NCAM).

In at least one embodiment, the current invention involves the use of a monoclonal antibody, neuro 4.1, directed against human L1, which can be used in any of the methods of the current invention. The neuro 4.1 antibody can recognize L1 and the common proteolytically-cleaved fragments thereof.

The NCAM 14.2 and/or neuro 4.1 antibody can be the entire monoclonal antibody or a functional fragment thereof. Functional fragments of antibodies include any portion of the antibody that is capable of binding the target antigen. As used herein, “fragment of an antibody” is used to mean a functional fragment. Antibody fragments specifically include F(ab′)₂, Fab, Fab′ and Fv fragments. These fragments can be generated from any class of antibody, but typically are made from IgG or IgM. They may be made by conventional recombinant DNA techniques or, using the classical method, by proteolytic digestion with papain or pepsin. See Current Protocols In Immunology, Chapter 2, Coligan et al., eds., (John Wiley & Sons 1991-92).

As used herein, the NCAM 14.2 antibody is an antibody, or fragment thereof, that possesses at least one V_{L} and at least one V_{H} chain of the fully in-tact monoclonal NCAM 14.2 antibody. In other words, “NCAM 14.2 antibody” includes the in-tact antibody and fragments thereof, as well as multispecific and multivalent antibodies, provided that the at least one of the binding sites of the multispecific or multivalent antibody comprises the V_{L} and V_{H} chains of the full length NCAM 14.2 antibody.

As used herein, the neuro 4.1 antibody is an antibody, or fragment thereof, that possesses at least one V_{L} and at least one V_{H} chain of the fully in-tact monoclonal neuro 4.1 antibody. In other words, “neuro 4.1 antibody” includes the in-tact antibody and fragments thereof, as well as multispecific and multivalent antibodies, provided that the at least one of the binding sites of the multispecific or multivalent antibody comprises the V_{L} and V_{H} chains of the full length neuro 4.1 antibody.

By “multispecific” is meant that the antibody, or fragment thereof, may bind simultaneously to at least two different targets having two different structures. By “multivalent” is meant that the antibody or fragments thereof may bind more than one target, which may or may have the same different structures, simultaneously. Thus, for example, the antibodies of the invention encompass an antibody or fragment thereof that binds NCAM and L1 simultaneously.

F(ab′)₂ fragments are typically about 110 kDa (IgG) or about 150 kDa (IgM) and contain two antigen-binding regions, joined at the hinge by disulfide bond(s). Virtually all, if not all, of the Fc fragment does not possess these antigen-binding sites. Fab′ fragments are typically about 55 kDa (IgG) or about 75 kDa (IgM) and can be formed, for example, by reducing the disulfide bond(s) of an F(ab′)₂ fragment. The resulting free sulfhydryl group(s) may be used to conjugate Fab′ fragments to other molecules, such as detection reagents (e.g., enzymes), other Fab′ or Fab fragments.

Fab fragments are monovalent and usually are about 50 kDa (from any source). Fab fragments include the light (L) and heavy (H) chain, variable (V_{L} and V_{H}, respectively) and constant (C_{L} and C_{H}, respectively) regions of the antigen-binding portion of the antibody. An intramolecular disulfide bridge links the H and L portions.

Fv fragments are typically about 25 kDa (regardless of source) and contain the variable regions of both the light and heavy chains (V_{L} and V_{H}, respectively). Usually, the V_{L} and V_{H} chains are held together only by non-covalent interacts and, thus, they readily dissociate. The V_{L} and V_{H} chains do, however, have the advantage of small size and they are able to retain similar, if not the same, binding properties of the larger Fab fragments. Accordingly, methods have been developed to crosslink the V_{L} and V_{H} chains, using, for example, glutaraldehyde (or other chemical crosslinkers), intermolecular disulfide bonds (by incorporation of cysteines) and peptide linkers. The resulting Fv is now a single chain Fv fragment (i.e., SCFV).

Alternatively, SCFvs may be generated by recombinant methods, allowing the generation of Fvs with new specificities by mixing and matching variable chains from different antibody sources. In a typical method, a recombinant vector would be provided which comprises the appropriate regulatory elements driving expression of a cassette region. The cassette region would contain a DNA encoding a peptide linker, with convenient sites at both the 5′ and 3′ ends of the linker for generating fusion proteins. The DNA encoding a variable region(s) of interest may be cloned in the vector to form fusion proteins with the linker, thus generating an SCFv.

In one embodiment, DNAs encoding two Fvs may be ligated to the DNA encoding the linker, and the resulting tripartite fusion may be ligated directly into a conventional expression vector. The SCFv DNAs generated any of these methods may be expressed in prokaryotic or eukaryotic cells, depending on the vector chosen.

The examples presented herein are meant to illustrate some of the embodiments of the current invention and are not intended to limit the scope of the claims in any way.

### EXAMPLES

### Example 1

### Patients and Collection of Cerebrospinal Fluid

218 patients with different neurological diseases, including Alzheimer disease (AD), vascular dementia (VD), dementia of mixed type (MT), diffuse Lewy body dementia (DLBD), multiple system atrophy (MSA), Parkinson disease (PD), major and minor depression, multiple sclerosis (MS), epilepsy, amyotrophic lateral sclerosis (ALS), polyneuropathy (PNP), progressive supranuclear palsy (PSP), Pick disease, hydrocephalus, schizophrenia, paranoia, epidural hematoma, cervical osteochondrosis, herpes zoster infection, spinal muscular atrophy and paraneoplastic encephalopathy were examined in this study. In addition, normal controls without any confirmed neurological or neuropsychiatric diseases were studied.

Clinical diagnoses were made according to published criteria: Alzheimer disease and dementia of mixed type,¹ vascular dementia,¹⁶ multiple sclerosis,¹⁷ multiple system atrophy,¹⁸ Parkinson disease,¹⁹ diffuse Lewy bodies dementia ²⁰ or to general neurological practice, e.g. epilepsy, amyotrophic lateral sclerosis or polyneuropathy.

Dementia was diagnosed by standard clinical procedures and the degree defined by the score in the Mini Mental State Examine (MMSE).²¹ A score of 30 to 27 points was classified as non-demented, 26 to 20 as mildly demented, 19 to 10 points as moderately demented, and 9 to 0 points as severely demented.

Normal controls underwent lumbar puncture to collect cerebrospinal fluid (CSF) for diagnostic purpose, for instance, to exclude subarachnoid hemorrhage, and revealed no signs for any disease of the central or peripheral nervous systems and were inconspicuous for protein levels, albumin quotient, cell count, oligoclonal bands, immunoglobulins, glucose, lactate, lysozyme, neuron-specific enolase and copper.

CSFs were collected by lumbar puncture between L4 and L5. Patients were in supine position for two hours before puncture. CSFs were taken in 1 ml fractions in polypropylene tubes and immediately frozen and stored at −80° C. until biochemical analyses were performed. Informed consent, according to the declaration of Helsinki, was obtained before lumbar puncture, and the study was approved by the Ethical Committee of the City of Hamburg.

### Example 2

### Measuring the Levels of L1 and NCAM

We developed and optimized a sensitive capture ELISA as follows: Purified monoclonal “capture” antibodies (10 μg/ml) neuro 4.1.1.3.3, 14.2 or 735 against L1, NCAM or PSA, respectively were pipetted into each well of 96-well microplates (NUNC Immuno Maxisorp FB, Roskilde, Denmark) and incubated overnight at 4° C. Plates were then washed five times with phosphate buffered saline (PBS), pH 7.4, containing 0.05% Tween 20, and blocked with a 5% solution of nonfat dry milk (Fluka, Deisenhofen, Germany) in PBS, pH 7.4, for 1.5 h at 37° C. Protein standards were human L1-Fc (ranging from 2 to 64 ng/ml), human NCAM-Fc (ranging from 9 to 300 ng/ml) and mouse PSA-NCAM (ranging from 1 to 50 ng/ml) and were freshly prepared for each test from a frozen stock solution in serial two-fold dilutions. Each well was then loaded with standard or CSF test sample (undiluted, 1:8 diluted or 1:15 diluted for L1, NCAM or PSA, respectively) and incubated for 1.5 h at room temperature. The “detector” antibodies (1.5 μg/ml) (polyclonal anti-human L1 in case of L1 or polyclonal anti-human NCAM 3731 for detection of both NCAM and PSA-NCAM) were loaded into each well and incubated for 20 h at room temperature. After washing, peroxidase-labeled goat anti-rabbit IgG antibody (0.16 μg/ml) was added into each well and incubated for 1.5 h at room temperature. ABTS (Roche Diagnostics, Mannheim, Germany) was added for calorimetric detection. The enzymatic reaction was stopped with 1.25% sodium fluoride, and absorbance values were measured at 405 nm. Each CSF sample was run in triplicate in a blinded fashion. A control series of standard proteins (L1-Fc, NCAM-Fc and PSA-NCAM) was included on every microtiter plate. All CSF samples were tested for each antigen.

The capture antibodies used in the ELISA were monoclonal anti-human L1 antibody neuro 4.1.1.3, monoclonal anti-human NCAM antibody NCAM 14.2 as well as the polyclonal anti-human NCAM antibody 3731 which were provided by BD Technologies, Research Triangle Park, N.C., USA. The polyclonal anti-human L1 antibodies were prepared in rabbits against human L1-Fc (Lab As Ltd, Tartu, Estonia). The anti-Fc antibodies were isolated from the serum of the rabbits by absorption on a human IgG Sepharose column.²² Human L1-Fc protein and human NCAM-Fc protein were produced as described previously ^{23,24}. Mouse monoclonal antibody 735 against α2,8-polysialic acid (PSA),²⁵ was a gift from Dr. Rita Gerardy-Schahn (Medizinische Hochschule, Hannover, Germany). The murine PSA-NCAM-Fc²⁶ was produced using a stably transfected TE671 cell line provided by Dr. G. Rougon (Laboratoire de Genetique et Physiologie du Developement, CNRS, Marseille, France). Peroxidase-labeled goat anti-rabbit IgG antibodies were from Dianova (Hamburg, Germany).

### Example 3

### Statistical Analysis

Comparisons between different groups were performed using the non-parametric Kruskal-Wallis test. The level of statistical significance was set at p<0.05. Normal distribution of CSF parameters for dementia/non-dementia and neurodegeneration/non-neurodegeneration groups was confirmed using the Kolmogorov-Smirnov test. Between-group differences in discontinuous variables were analyzed by Student's t-test for independent groups. Correlations were analyzed by Pearson Product Moment Correlations. ANOVA with age as co-variant was used to explore the extent to which values were specifically influenced by age. Post-hoc analysis was done with the Scheffe-test. Additionally, we estimated the influence of presence of dementia, neurodegeneration, gender and age on L1, NCAM and polysialic acid by multiple regression analysis including measurement of partial correlation of independent variables. Results are expressed as mean±standard error.

Results

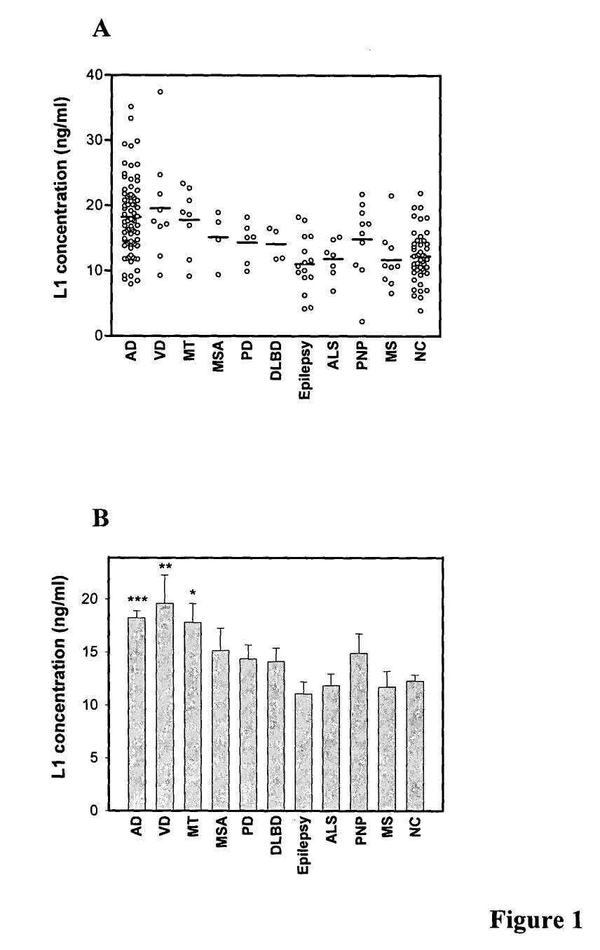

Statistical analysis shows an increase (p<0.0001) in mean levels of L1 in the cerebrospinal fluid (CSF) of Alzheimer's disease (AD) patients (18.2±0.7 ng/ml, n—76), when compared to normal controls (12.2±0.6 ng/ml, n=46) (FIG. 1). In FIG. 1A, each horizontal line represents the mean value of L1 concentration. FIG. 1B depicts the mean values of L1 concentration±standard error. Statistical analysis (with correlation for age) shows a higher level (***p<0.0001) of L1 in the CSF of AD patients, compared to the levels found in normal controls. There was also an increase (p<0.01 and p<0.05, respectively) in L1 levels in patients with vascular dementia (VD) (19.6±2.7 ng/ml, n=9) and dementia of mixed type (MT) (17.8±1.8 ng/ml, n=8), compared to normal controls. Elevated L1 levels in the CSF of these groups were observed irrespective of age and gender. Other groups: multiple system atrophy (n=4), Parkinson disease (n=6), diffuse Lewy body dementia (n=4), epilepsy (n=15), amyotrophic lateral sclerosis (n=7), polyneuropathy (n=10), multiple sclerosis (n=9), did not show any difference in comparison to the normal control group.

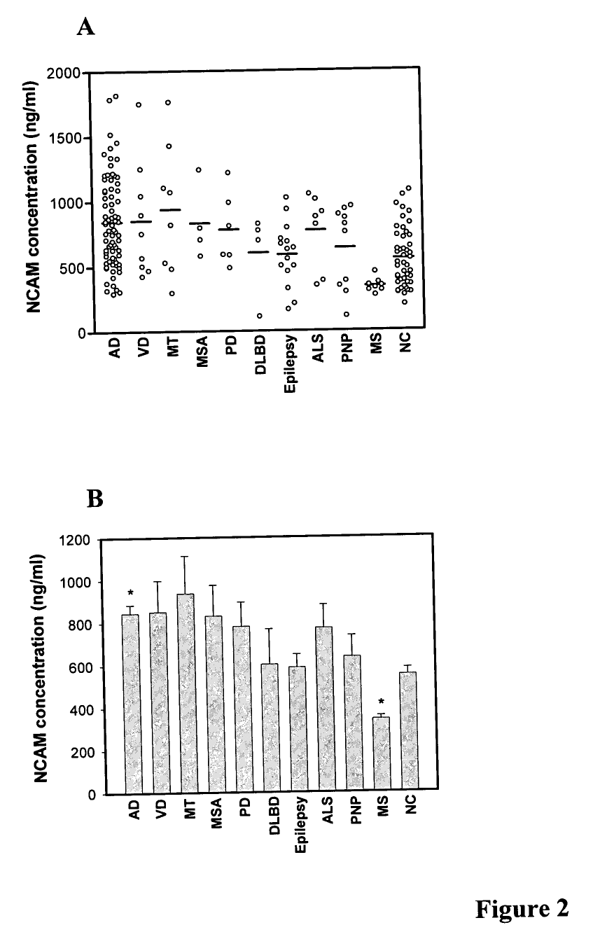

Statistical analysis shows an increase in the mean levels of NCAM (p<0.0001) in the CSF of AD patients (845.4±39.7 ng/ml, n=76), compared to normal controls (551.3±33.3 ng/ml, n=46). Furthermore, levels of NCAM were higher (p<0.05) in patients with vascular dementia (852.1±146.2, n=9) and dementia of mixed type (937.5±177.4, n=8) in comparison to normal controls (FIG. 2). In FIG. 2A, each horizontal line represents the mean value of NCAM concentration. FIG. 2B depicts the mean values of NCAM concentration±standard error. NCAM concentration was independent of gender, but was correlative with age. When corrected for age as a co-variable, the association of higher NCAM concentrations in the patients with AD remained significant (p<0.05). However, when patients with vascular dementia (VD) and dementia of mixed type (MT) were compared to the normal controls, with correction for age, the difference was no longer significant. NCAM levels were decreased (p<0.05) in the CSF from patients with multiple sclerosis (342.2±16.7 ng/ml, n=9) in comparison to normal controls, and the difference remained significant after correction for age.

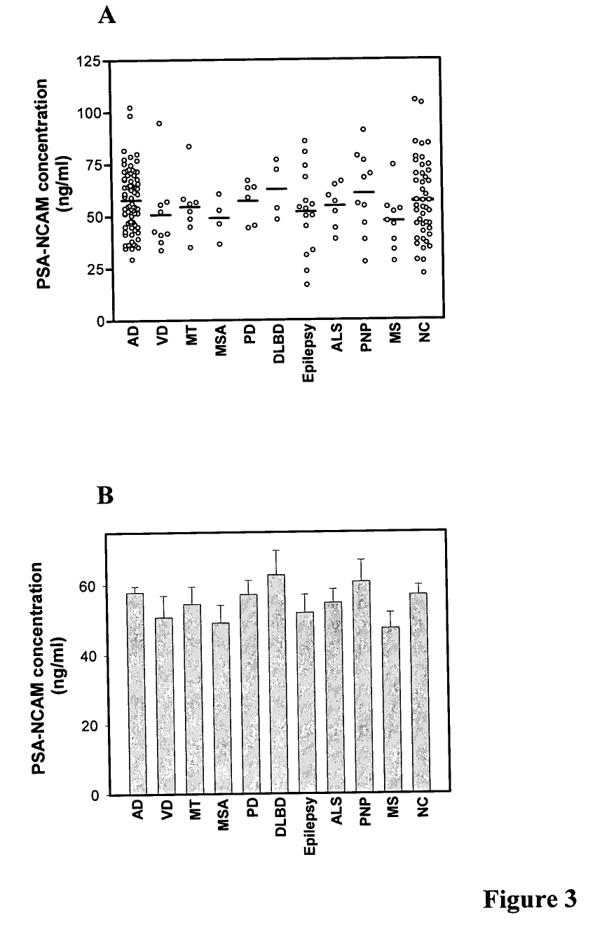

Levels of PSA-NCAM in the CSF did not differ among the groups (FIG. 3). In FIG. 3A, each horizontal line represents the mean value of PSA-NCAM concentration. FIG. 3B depicts the mean values of PSA-NCAM concentration±standard error. The mean concentration of PSA-NCAM in the normal control group was 56.9±2.8 ng/ml (n=46).

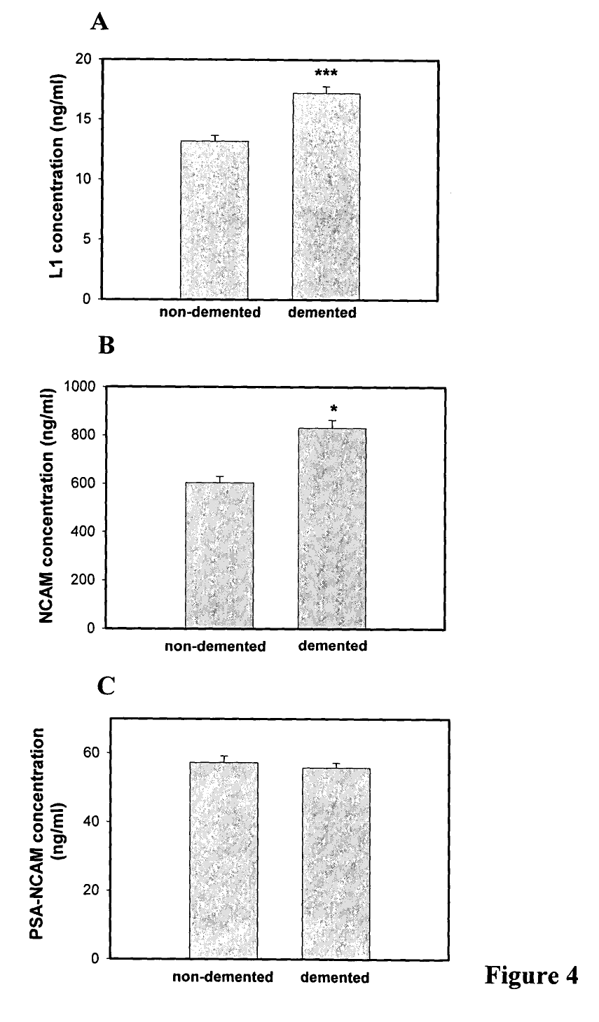

To investigate whether the levels of adhesion molecules are associated with all forms of dementia, we further analyzed the concentrations of L1, NCAM and PSA-NCAM in patients after their assignment to either a dementia and non-dementia group. Statistical analysis revealed that there was an increase (p<0.000001) in mean levels of L1 in the CSF of patients with dementia (17.2±0.5 ng/ml, n=117), compared to the non-dementia group (13.2±0.5 ng/ml, n=101) (FIG. 4A). The L1 concentration in these groups was independent of gender but correlated with age (r=0.28; p<0.05). However, the association of higher L1 concentrations in patients with all forms of dementia remained when corrected for age as co-variable (p<0.00001).

When corrected for age within the group of demented patients, the severity of dementia as defined by the Mini Mental State Examine (MMSE) as a discontinuous variable (stepwise regression), or the assignment of patients to groups of mildly (n=68), moderately (n=42) and severely (n=7) demented patients did not correlate with the L1 concentration (data not shown).

However, NCAM concentration was higher (p<0.00001) in the CSF of patients with dementia (830.3±33.3 ng/ml, n=117), compared to the non-dementia group (603.5±26.0 ng/ml, n=101) (FIG. 4B). NCAM concentrations were independent of gender but correlated with age (r=0.37; p<0.05). The association of higher NCAM concentrations with dementia was still present when corrected for age as co-variable (p<0.05).

When corrected for age within the group of demented patients, the degree of dementia as defined by the MMSE as a discontinuous variable (stepwise regression), or the assignment of patients to groups of mildly (n=71), moderate (n=42) and severely (n=7) demented did not correlate with the NCAM concentration.

Statistical analysis did not reveal any difference in the concentrations of PSA-NCAM in the CSF of demented and of non-demented patients (FIG. 4C). The mean concentration of PSA-NCAM in patients without dementia was 57.3±1.9 ng/ml (n—102), while the mean concentration of PSA-NCAM in patients with dementia was 55.7±1.3 ng/ml (n=116). Concentrations of PSA-NCAM were independent of gender and did not correlate with age.

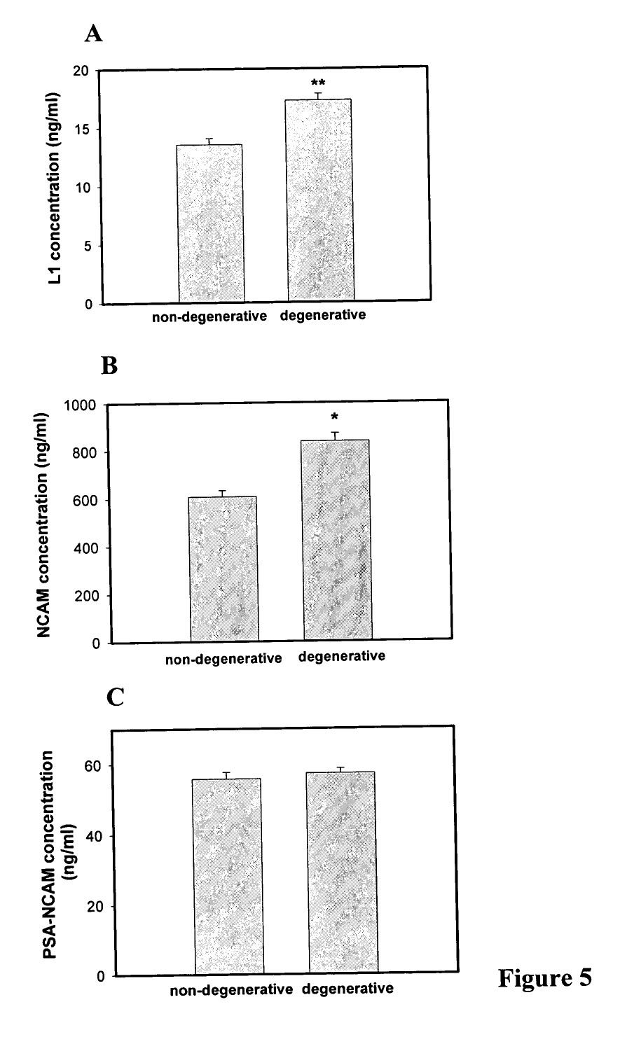

In the majority of patients, dementia is associated with neurodegeneration. Thus, we explored whether neurodegenerative processes in the central nervous system can be correlated with elevated L1 and NCAM concentrations. The group of conditions classified as neurodegenerative in this study included AD (n=76), dementia of mixed type (n=8), Parkinson disease (n=6), diffuse Lewy body dementia (n=4), multiple system atrophy (n=4), amyotrophic lateral sclerosis (n=7), progressive supranuclear palsy (n=1), and Pick disease (n—2). The group of conditions classified as non-degenerative in this study include vascular dementia (n=9), multiple sclerosis (n=9), epilepsy (n=15), polyneuropathy (n=10), normal pressure hydrocephalus (n=2), psychosis (n=3), alcoholism (n=3), major depression (n=6), paraneoplastic encephalopathy (n=1), rheumatoid arthrosis (n—), epidural hematoma (n=2), varicella zoster neuritis (n=2), spinal muscle atrophy (n=1), and normal controls (n=46). Patients with neurodegenerative diseases had significantly higher (p<0.000001) concentrations of L1 in the CSF (17.3±0.5 ng/ml, n=108), compared to the group of patients with non-degenerative diseases (13.5±0.5 ng/ml, n=110) (FIG. 5A). This difference remained significant when corrected for age (p<0.001). However, when only those patients with neurodegenerative diseases were compared to the group of patients with non-degenerative diseases without dementia, the difference was no longer significant (data not shown).

The levels of NCAM were higher (p<0.000001) in the CSF of patients with neurodegenerative disorders (837.8±33.1 ng/ml, n=108) compared patients with non-degenerative diseases, including normal controls (606.9±26.3 ng/ml, n=110) (FIG. 5B). This difference remained significant (p<0.05) when corrected for age. As shown for L1, NCAM values were not different between non-demented patients with neurodegenerative diseases and non-demented patients with non-degenerative diseases (data not shown).

The average concentration of PSA-NCAM in patients with neurodegenerative diseases (57.3±1.4 ng/ml) was not statistically different from patients with non-degenerative diseases, including controls (55.8±1.9 ng/ml) (FIG. 5C).

Additionally, we performed a stepwise multiple regression analysis with L1 as a dependent variable, and age, gender, dementia and neurodegenerative etiology as independent variables. This stepwise analysis confirmed the significant influence of dementia (beta 0.175, correlation 0.30) and neurodegeneration (beta 0.165, correlation 0.29), on levels of L1 (data not shown). However, this analysis also demonstrated that age (beta 0.085, correlation 0.26) and gender (beta −0.01, correlation 0.05) do not have a significant influence on the levels of L1 (data not shown). Partial correlation indicates that L1 was almost equally influenced by dementia (beta 0.14, p=0.049) and neurodegeneration (beta 0.14, p=0.047), without influence by age (beta 0.07, p=0.34) and gender (beta −0.01, correlation 0.05, p=0.9) (data not shown).

The stepwise multiple regression analysis for NCAM confirmed a significant influence of age (beta 0.23, correlation 0.35) and neurodegeneration (beta 0.18, correlation 0.33) (data not shown). However, this stepwise analysis revealed no influence of dementia (beta 0.112, correlation 0.30) and gender (beta −0.07, correlation 0.01) on the levels of NCAM (data not shown). Partial correlation indicates that NCAM was influenced more by age (beta 0.21, p=0.004) and neurodegeneration (beta 0.17, p=0.02) than by dementia (beta 0.10, p=0.18) and gender (beta −0.07, p=0.32) (data not shown).

Stepwise multiple regression analysis did not reveal any influence of age, gender, dementia or neurodegeneration on the levels of PSA-NCAM (data not shown).

### Example 4

### Production of NCAM 14.2 Antibody

Leu19, an anti-NCAM monoclonal antibody (Becton Dickinson Advanced Cellular Biology, San Jose, Calif.) was used to prepare an immunoaffinity column. NCAM from human adult brain was purified by standard techniques using the Leu19 affinity column. Using conventional techniques for preparation of monoclonal antibodies, mice were immunized with the immunoaffinity purified NCAM and their spleen cells were subsequently purified and fused with murine myeloma cells. The resulting hybridomas were screened for reactivity with the NCAM in ELISA assays and rescreened with synthetic peptides representing various immunoglobulin domains of NCAM. The hybridoma which produces NCAM 14.2 was selected for further characterization based on its strong reactivity with the immunogen. The NCAM 14.2 antibody is described in Lanier, L. L., et al., J. Immunology, 146:4421-4426 (1991), which is hereby incorporated by reference.

### Example 5

### Production of Neuro 41 Antibody

Using conventional techniques for preparation of monoclonal antibodies, mice were immunized with a crude human brain extract and their spleen cells were subsequently purified and fused with murine myeloma cells. The resulting hybridomas were screened for reactivity with the L1 in ELISA assays and rescreened with synthetic peptides representing various immunoglobulin domains of L1. The hybridoma which produces neuro 4.1 was selected for further characterization based on its strong reactivity with the immunogen. The neuro 4.1 antibody is described in Poltorak, M., et al., Experimental Neurology, 131:266-272 (1995), which is hereby incorporated by reference.

1. A method of diagnosing a dementia-related neurological disorder in a patient exhibiting symptoms of said dementia-related neurological disorder and suspected of having said dementia-related neurological disorder comprising
a) obtaining a sample from said patient;
b) determining the levels of at least one cell adhesion molecule selected from the group consisting of L1 and neural cell adhesion molecule (NCAM); and
c) diagnosing said dementia-related neurological disorders in said patient exhibiting said symptoms,
wherein the basis of said diagnosis is that said determined levels of said at least one cell adhesion molecule in b) are greater than the levels of said at least one cell adhesion molecule in a patient not exhibiting symptoms of said dementia-related neurological disorder and not suspected of having said dementia-related neurological disorder.

2. The method of claim 1, wherein the levels of L1 are determined.

3. The method of claim 2, wherein said dementia-related neurological disorder is selected from the group consisting of vascular dementia and dementia of mixed type.

4. The method of claim 1, wherein the levels of NCAM are determined.

5. The method of claim 4, wherein said dementia-related neurological disorder is selected from the group consisting of Alzheimer's Disease (AD) and multiple sclerosis (MS).

6. The method of claim 1, wherein the levels of both L1 and NCAM are determined.

7. The method of claim 6, wherein said dementia-related neurological disorder is selected from the group consisting of Alzheimer's Disease (AD) and multiple sclerosis (MS).

8. The method of claim 1, 4 or 6, wherein said determining said levels of NCAM comprises the NCAM 14.2 antibody.

9. The method of claim 1, 2 or 6, wherein said determining the levels of L1 comprises the neuro 4.1 antibody.

10. A method of monitoring the progression of a dementia-related neurological disorder in a patient being diagnosed with said dementia-related neurological disorder comprising
a) obtaining a first sample from said patient;
b) determining the levels in said first sample of at least one cell adhesion molecule selected from the group consisting of L1 and neural cell adhesion molecule (NCAM);
c) obtaining a second sample from said patient; and
d) determining the levels in said second sample of at least one cell adhesion molecule selected from the group consisting of L1 and neural cell adhesion molecule (NCAM); and
e) comparing said determined levels in b) with said determined levels in d) to reveal a difference in said determined levels;
wherein said difference indicates the progression of said dementia-related neurological disease in said patient.

11. The method of claim 10, wherein the levels of L1 are determined.

12. The method of claim 11, wherein said dementia-related neurological disorder is selected from the group consisting of vascular dementia and dementia of mixed type.

13. The method of claim 10, wherein the levels of NCAM are determined.

14. The method of claim 13, wherein said dementia-related neurological disorder is selected from the group consisting of Alzheimer's Disease (AD) and multiple sclerosis (MS).

15. The method of claim 10, wherein the levels of both L1 and NCAM are determined.

16. The method of claim 15, wherein said dementia-related neurological disorder is selected from the group consisting of Alzheimer's Disease (AD) and multiple sclerosis (MS).

17. The method of claim 10, 13 or 15, wherein said determining said levels of NCAM comprises the NCAM 14.2 antibody.

18. The method of claim 10, 11 or 15, wherein said determining said levels of L1 comprises the neuro 4.1 antibody.
